(19) United States
(12) Patent Application Publication
(10) Pub. No.: US 2008/0194405 A1
(43) Pub. Date: Aug. 14, 2008

(54) Fungicidal 5-Hydroxypyrazolines, Method for Producing the Same and Agents Comprising the Same

(75) Inventors: Markus Gewehr, Kastellaun (DE); Jochen Dietz, Mannheim (DE); Thomas Grote, Wachenheim (DE); Andreas Gypser, Mannheim (DE); Matthias Niedenbruck, Limburgerhof (DE)

Correspondence Address: HUTCHISON LAW GROUP PLLC, PO BOX 31686, RALEIGH, NC, 27612, US

(73) Assignee: BASF Atkiengesellschaft, Ludwigshafen (DE)

(21) Appl. No.: 11/915,989

(22) PCT Filed: May 24, 2006
(86) PCT No.: PCT/EP2006/062565

§ 371 Date: Nov. 29, 2007

(30) Foreign Application Priority Data
May 31, 2005 (DE) 102005025397.0

(51) Int. Cl. A01N 43/56; C07D 231/08; A01P 3/00

(52) U.S. Cl. 504/100; 514/403; 548/366.1; 548/370.4

(57) ABSTRACT

The present invention relates to novel 5-hydroxypyrazolines of the formula I

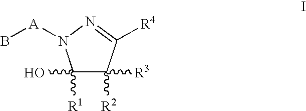

in which the substituents are as defined below:
B is phenyl, naphthyl or 5- or 6-membered hetaryl which contains one to four heteroatoms from the group consisting of O, N and S;
A is C═O, C═S or SO₂;
R¹ is alkyl, haloalkyl, alkenyl, haloalkenyl, alkynyl or haloalkynyl, cycloalkyl, cycloalkenyl, cycloalkynyl, phenyl, 5- or 6-membered heterocyclyl or hetaryl which contains one to four heteroatoms from the group consisting of O, N and S;
R² is alkyl, haloalkyl, alkenyl, haloalkyl, alkynyl, haloalkynyl, cycloalkyl, cycloalkenyl, cycloalkynyl, phenyl, 5- or 6-membered hetaryl or heterocyclyl which contains one to four heteroatoms from the group consisting of O, N and S;
R³ is hydrogen, nitro, cyano, alkyl, haloalkyl, alkoxy, haloalkoxy, alkenyl, haloalkenyl, alkynyl, haloalkynyl or NR′₂, where
   R′ independently of one another are hydrogen or alkyl;
R⁴ is hydrogen, halogen, nitro, cyano, NR′₂, alkyl, haloalkyl, COOR′ or 5- or 6-membered hetaryl or heterocyclyl;
   where the variables mentioned above may be substituted according to the description;
   processes for their preparation, their use for controlling harmful fungi and compositions comprising them.

The present invention relates to novel 5-hydroxypyrazolines of the formula I

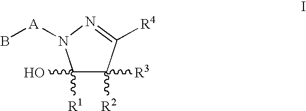

in which the substituents are as defined below:
B is phenyl, naphthyl or 5- or 6-membered hetaryl which contains one to four heteroatoms from the group consisting of O, N and S;
A is C═O, C═S or SO₂;
R¹ is C₂-C₁₀-alkyl, C₁-C₁₀-haloalkyl, C₃-C₁₀-alkenyl, C₃-C₁₀-haloalkenyl, C₃-C₁₀-alkynyl or C₃-C₁₀-haloalkynyl, C₃-C₁₀-cycloalkyl, C₃-C₁₀-cycloalkenyl, C₈-C₁₀-cycloalkynyl, phenyl, 5- or 6-membered heterocyclyl or hetaryl which contains one to four heteroatoms from the group consisting of O, N and S;
R² is C₁-C₁₀-alkyl, C₁-C₁₀-haloalkyl, C₂-C₁₀-alkenyl, C₂-C₁₀-haloalkyl, C₃-C₁₀-alkynyl, C₃-C₁₀-haloalkynyl, C₃-C₁₀-cycloalkyl, C₃-C₁₀-cycloalkenyl, C₃-C₁₀-cycloalkynyl, phenyl, 5- or 6-membered hetaryl or heterocyclyl which contains one to four heteroatoms from the group consisting of O, N and S;
R³ is hydrogen, nitro, cyano, C₁-C₄-alkyl, C₁-C₄-haloalkyl, C₁-C₄-alkoxy, C₁-C₄-haloalkoxy, C₂-C₄-alkenyl, C₂-C₄-haloalkenyl, C₂-C₄-alkynyl or C₂-C₄-haloalkynyl or NR′₂, where
   R′ independently of one another are hydrogen or C₁-C₄-alkyl;
R⁴ is hydrogen, halogen, nitro, cyano, NR′₂, C₁-C₄-alkyl, C₁-C₄-haloalkyl, COOR′ or 5- or 6-membered hetaryl or heterocyclyl;
   where the variables mentioned above may be partially or fully halogenated and/or may carry one to four groups R^{a},
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, formyl, C₁-C₆-alkylthio, C₁-C₆-alkylamino, di-C₁-C₆-alkylamino, C₂-C₈-alkenyl, C₂-C₈-haloalkenyl, C₃-C₈-cycloalkenyl, C₂-C₆-alkenyloxy, C₃-C₆-haloalkenyloxy, C₂-C₆-alkynyl, C₂-C₆-haloalkynyl, C₃-C₆-alkynyloxy, C₃-C₆-haloalkynyloxy, C₃-C₆-cycloalkoxy, C₃-C₆-cycloalkenoxy, C₁-C₃-oxyalkylenoxy, phenyl, naphthyl, a five- to ten-membered saturated, partially unsaturated or aromatic heterocycle which contains one to four heteroatoms from the group consisting of O, N and S, CRⁱⁱⁱ═NOR^{iv}, where
   Rⁱⁱⁱ is hydrogen, alkyl, cycloalkyl or aryl and
   R^{iv} is alkyl, alkenyl, haloalkenyl, alkynyl or arylalkyl, or
   NR^{v}—CO-D-R^{vi}, where
   R^{v} is hydrogen, hydroxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-alkoxy-C₁-C₆-alkyl, C₁-C₆-alkoxy-C₁-C₆-alkoxy or C₁-C₆-alkoxycarbonyl,
   R^{vi} is hydrogen, C₁-C₆-alkyl, C₂-C₆-alkenyl, C₂-C₆-alkynyl, C₃-C₆-cycloalkyl, C₃-C₆-cycloalkenyl, phenyl, phenyl-C₁-C₆-alkyl, hetaryl or hetaryl-C₁-C₆-alkyl and D is a direct bond, oxygen or nitrogen, where the nitrogen may carry one of the groups mentioned under R^{vi},
   where the aliphatic, alicyclic or aromatic groups R^{a} for their part may be partially or fully halogenated or may carry one to three groups R^{b}:
   R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, where the alkyl groups in these radicals contain 1 to 6 carbon atoms and the alkenyl or alkynyl groups mentioned in these radicals contain 2 to 8 carbon atoms;
      and/or one to three of the following radicals:
      cycloalkyl, cycloalkoxy, heterocyclyl, heterocyclyloxy, where the cyclic systems contain 3 to 10 ring members; phenyl, phenoxy, phenylthio, phenyl-C₁-C₆-alkoxy, phenyl-C₁-C₆-alkyl, hetaryl, hetaryloxy, hetarylthio, where the hetaryl groups contain 5 or 6 ring members, and
   the cyclic systems may be partially or fully halogenated or substituted by alkyl or haloalkyl groups.

Moreover, the invention relates to processes for their preparation, to their use for controlling harmful fungi and to compositions comprising them.

Substituted pyrazolin-5-ones having herbicidal and fungicidal activity are known from DE-A 37 28 278, fungicidally active 3-arylpyrazoles are disclosed in WO-A 94/29276 and WO 00/20399.

However, in many cases their activity is unsatisfactory. Accordingly, it was an object of the present invention to provide compounds having improved activity.

We have found that this object is achieved by the novel 5-hydroxypyrazoles of the formula I. The novel compounds differ from the compounds known from WO 00/20399 by the substitution in the 4-position.

Compounds of the formula I are present in a tautomeric equilibrium with the open-chain form Ia [cf.: J. Org. Chem. USSR (1983), 2037; ibid. (1984), 1247].

The invention therefore relates to both forms, even if, for the sake of clarity, only the ring form I is mentioned in each case.

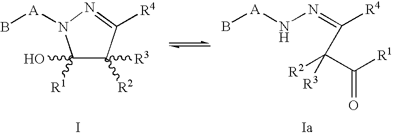

Compounds of the formula I in which A is C═O (formula IA) can be obtained, for example, by the following route:

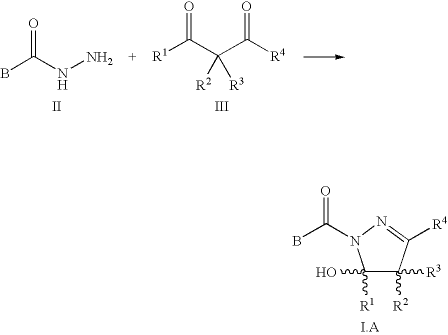

This reaction is usually carried out at from 0° C. to 200° C., preferably from 20° C. to 100° C., in an inert organic solvent [J. Org. Chem. USSR (Engl. Transl.), 16 (1980), 371; ibid. 21 (1985), 2279; ibid. 22 (1986), 250; ibid. 23 (1987), 1291; Indian J. Chem. Sect. B, 29 (1990), 887; Bull. Soc. Chem. Jp. 62 (1989), 3409].

Suitable solvents are aliphatic hydrocarbons, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane and tetrahydrofuran, nitriles, such as acetonitrile and propionitrile, alcohols, such as methanol, ethanol, n-propanol, isopropanol, n-butanol and tert-butanol, and also dimethyl sulfoxide, dimethylformamide and dimethylacetamide, particularly preferably methanol, ethanol and tetrahydrofuran. It is also possible to use mixtures of the abovementioned solvents.

The starting materials are generally reacted with one another in equimolar amounts. In terms of yield, it may be advantageous to employ an excess of III, based on II.

The hydrazides of the formula II required for preparing the compounds I are known from the literature [cf. J. Heterocycl. Chem. 16 (1976), 561; Helv. Chim. Acta, 27 (1944), 883; J. Chem. Soc. (1943), 413], or they can be prepared in accordance with the literature cited.

Hydrazides of the formula II are usually prepared from the corresponding carboxylic esters of the formula V by reaction with hydrazine hydrate. In the formula V, R′ is C₁-C₄-alkyl.

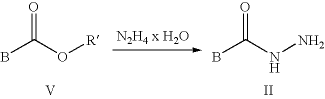

This reaction is usually carried out at from 0° C. to 150° C., preferably from 20° C. to 100° C., in an inert organic solvent [cf. J. Heterocycl. Chem. 16 (1976), 561; Helv. Chim. Acta, 27 (1944), 883; J. Chem. Soc. (1943), 413].

The diketones of the formula III required for preparing the compounds I are also known from the literature [Organikum, VEB Verlag der Wissenschaften, 15th ed. p. 584ff., Berlin 1976], or they can be prepared in accordance with the literature cited.

The compounds of the formula I in which A is SO₂ (formula I.B1) are preferably obtained by the following route:

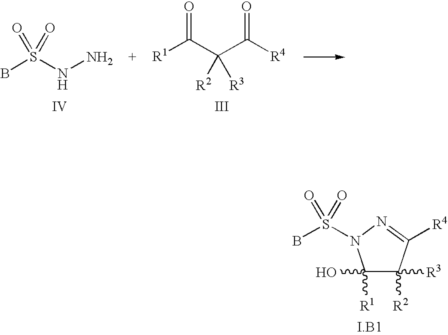

This reaction is advantageously carried out under the conditions given for the preparation of the compounds IA.

The starting materials are generally reacted with one another in equimolar amounts. In terms of yield, it may be advantageous to use an excess of III, based on IV.

The sulfonyl hydrazides of the formula IV required for preparing the compounds are known from the literature [J. Chem. Soc. Chem. Commun. (1972) 1132; J. Chem. Soc. (1949) 1148; Helv. Chim. Acta 42 (1962), 996] or they can be prepared in accordance with the literature cited.

The compounds of the formula I in which A is C═S (formula I.B2) can be obtained from the corresponding compounds of the formula I.A by reaction with a sulfurizing agent.

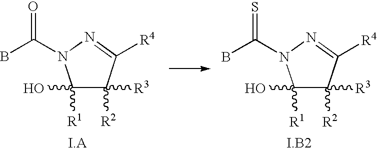

The sulfurization of I.A is carried out under conditions known per se, usually at from 0° C. to 180° C., preferably from 20° C. to 140° C., in an inert organic solvent [cf. Liebigs Ann. Chem., (1989), 177].

Suitable solvents are aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, and also dimethyl sulfoxide, particularly preferably toluene and tetrahydrofuran. It is also possible to employ mixtures of the abovementioned solvents.

Suitable sulfurizing agents are, for example, phosphorus pentasulfide or Lawesson's reagent.

The reaction mixtures are worked up in a customary manner, for example by mixing with water, phase separation and, if appropriate, chromatographic purification of the crude products. Some of the intermediates and end products are obtained in the form of colorless or slightly brownish, viscous oils, which are purified or freed from volatile components under reduced pressure and at moderately elevated temperatures. If the intermediates and end products are obtained as solids, purification can also be carried out by recrystallization or digestion.

In the definitions of the symbols given in the above formulae, collective terms were used which generally represent the following substituents:

halogen: fluorine, chlorine, bromine and iodine;
alkyl: saturated, straight-chain or branched hydrocarbon radicals having 1 to 4, 6, 8 or 10 carbon atoms, for example C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;
haloalkyl: straight-chain or branched alkyl groups having 1 to 10 carbon atoms (as mentioned above), where the hydrogen atoms in these groups may be partially or fully replaced by halogen atoms as mentioned above, for example C₁-C₂-haloalkyl such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl;
alkenyl: unsaturated, straight-chain or branched hydrocarbon radicals having 2 to 4, 6, 8 or 10 carbon atoms and a double bond in any position, for example C₂-C₆-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
haloalkenyl: unsaturated, straight-chain or branched hydrocarbon radicals having 2 to 10 carbon atoms and a double bond in any position (as mentioned above), where the hydrogen atoms in these groups may be partially or fully replaced by halogen atoms as mentioned above, in particular by fluorine, chlorine and bromine;
alkynyl: straight-chain or branched hydrocarbon groups having 2 to 4, 6, 8 or 10 carbon atoms and a triple bond in any position, for example C₂-C₆-alkynyl such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
haloalkynyl: unsaturated, straight-chain or branched hydrocarbon radicals having 2 to 10 carbon atoms and a triple bond in any position (as mentioned above), where the hydrogen atoms in these groups may be partially or fully replaced by halogen atoms as mentioned above, in particular fluorine, chlorine and bromine;
cycloalkyl: monocyclic, saturated hydrocarbon groups having 3 to 6, 8, 10 or 12 carbon ring members, for example C₃-C₈-cycloalkyl such as cyclopropyl, cyclobutyl, cyclopentyl, cyclohexyl, cycloheptyl and cyclooctyl;
heterocyclyl: 5- or 6-membered heterocycles containing, in addition to carbon ring members, one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydro-triazin-2-yl and 1,2,4-hexahydrotriazin-3-yl;
heteroaryl:
5-membered heteroaryl containing one to four nitrogen atoms or one to three nitrogen atoms and one sulfur or oxygen atom: heteroaryl groups having 5 ring members which, in addition to carbon atoms, may contain one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;
benzo-fused 5-membered heteroaryl containing one to three nitrogen atoms or one nitrogen atom and one oxygen or sulfur atom: heteroaryl groups having 5 ring members which, in addition to carbon atoms, may contain one to four nitrogen atoms or one to three nitrogen atoms and one sulfur or oxygen atom as ring members and in which two adjacent carbon ring members or one nitrogen and one adjacent carbon ring member may be bridged by a buta-1,3-diene-1,4-diyl group;
5-membered heteroaryl which is linked via nitrogen and contains one to four nitrogen atoms, or benzo-fused 5-membered heteroaryl which is linked via nitrogen and contains one to three nitrogen atoms: heteroaryl groups having 5 ring members which, in addition to carbon atoms, may contain one to four nitrogen atoms or one to three nitrogen atoms as ring members and in which two adjacent carbon ring members or one nitrogen and one adjacent carbon ring member may be bridged by a buta-1,3-diene-1,4-diyl group, these rings being linked to the skeleton via one of the nitrogen ring members;
6-membered heteroaryl containing one to three or one to four nitrogen atoms: heteroaryl groups having 6 ring members which, in addition to carbon atoms, may contain one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

In general, the alkyl groups in the various substituents preferably have 1 to 6 carbon atoms, in particular 1 to 4 carbon atoms, and the alkenyl or alkynyl groups mentioned contain 2 to 8 carbon atoms.

With respect to the intended use of the 5-hydroxypyrazolines of the formula I, particular preference is given to the following meanings of the substituents, in each case both on their own or in combination:

Particular preference is given to compounds I.A.

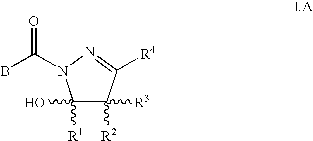

In another embodiment of the compounds of the formula I, A is SO₂.

In another embodiment of the compounds of the formula I, A is C═S.

Preference is given to compounds of the formula I in which R¹ is not methyl. Particular preference is given to compounds I in which R¹ is C₂-C₄-alkyl, in particular C₃-C₄-alkyl.

In addition, particular preference is given to compounds I in which R¹ is C₁-C₄-haloalkyl.

Particular preference is also given to compounds I in which R¹ is C₃F₇ or C₂F₅.

Moreover, particular preference is given to compounds I in which R¹ is optionally para-substituted phenyl.

Moreover, particular preference is given to compounds I in which B is phenyl which carries a substituent in the 3-position.

Moreover, particular preference is given to compounds I in which B is phenyl which carries a substituent in the 4-position.

Furthermore, particular preference is given to compounds I in which B is phenyl which carries a substituent in the 3,4-position.

In addition, particular preference is given to compounds I in which B is heteroaryl.

The invention preferably provides compounds of the formula I in which R² is C₁-C₄-alkyl, in particular methyl or ethyl.

Furthermore, particular preference is given to compounds I in which R³ is hydrogen.

Moreover, particular preference is given to compounds I in which R⁴ is hydrogen or methyl.

Likewise, particular preference is given to compounds I in which R⁴ is trifluoromethyl.

Particular preference is also given to compounds I in which R⁴ is cyano, C₂-C₄-alkyl or C₂-C₄-haloalkyl.

With respect to their use, particular preference is given to the compounds I compiled in the tables below. Moreover, the groups mentioned for a substituent in the tables are, by themselves and independently of the combination in which they are mentioned, a particularly preferred embodiment of the substituent in question.

### Table 1

Compounds of the formula I.A in which R¹ is C₂F₅, R² is methyl, R³ is hydrogen, R⁴ is methyl and B for a compound corresponds in each case to one row of Table A

### Table 2

Compounds of the formula I.A in which R¹ is C₂F₅, R² is methyl, R³ is hydrogen, R⁴ is trifluoromethyl and B for a compound corresponds in each case to one row of Table A

### Table 3

Compounds of the formula I.A in which R¹ is C₃F₇, R² is methyl, R³ is hydrogen, R⁴ is methyl and B for a compound corresponds in each case to one row of Table A

### Table 4

Compounds of the formula I.A in which R¹ is C₃F₇, R² is methyl, R³ is hydrogen, R⁴ is trifluoromethyl and B for a compound corresponds in each case to one row of Table A

### Table 5

Compounds of the formula I.A in which R¹ is C₄F₉, R² is methyl, R³ is hydrogen, R⁴ is methyl and B for a compound corresponds in each case to one row of Table A

### Table 6

Compounds of the formula I.A in which R¹ is C₄F₉, R² is methyl, R³ is hydrogen, R⁴ is trifluoromethyl and B for a compound corresponds in each case to one row of Table A

### Table 7

Compounds of the formula I.A in which R¹ is 4-Cl—C₆H₅, R² is methyl, R³ is hydrogen, R⁴ is methyl and B for a compound corresponds in each case to one row of Table A

### Table 8

Compounds of the formula I.A in which R¹ is 4-Cl—C₆H₅, R² is methyl, R³ is hydrogen, R⁴ is trifluoromethyl and B for a compound corresponds in each case to one row of Table A

### Table 9

Compounds of the formula I.A in which R¹ is 4-F—C₆H₅, R² is methyl, R³ is hydrogen, R⁴ is methyl and B for a compound corresponds in each case to one row of Table A

### Table 10

Compounds of the formula I.A in which R¹ is 4-F—C₆H₅, R² is methyl, R³ is hydrogen, R⁴ is trifluoromethyl and B for a compound corresponds in each case to one row of Table A

### Table 11

Compounds of the formula I.A in which R¹ is 4-Br—C₆H₅, R² is methyl, R³ is hydrogen, R⁴ is methyl and B for a compound corresponds in each case to one row of Table A

### Table 12

Compounds of the formula I.A in which R¹ is 4-Br—C₆H₅, R² is methyl, R³ is hydrogen, R⁴ is trifluoromethyl and B for a compound corresponds in each case to one row of Table A

### Table 13

Compounds of the formula I.A in which R¹ is 4-CH₃—C₆H₅, R² is methyl, R³ is hydrogen, R⁴ is methyl and B for a compound corresponds in each case to one row of Table A

### Table 14

Compounds of the formula I.A in which R¹ is 4-CH₃—C₆H₅, R² is methyl, R³ is hydrogen, R⁴ is trifluoromethyl and B for a compound corresponds in each case to one row of Table A

### Table 15

Compounds of the formula I.A in which R¹ is C₂F₅, R² is ethyl, R³ is hydrogen, R⁴ is methyl and B for a compound corresponds in each case to one row of Table A

### Table 16

Compounds of the formula I.A in which R¹ is C₂F₅, R² is ethyl, R³ is hydrogen, R⁴ is trifluoromethyl and B for a compound corresponds in each case to one row of Table A

### Table 17

Compounds of the formula I.A in which R¹ is C₃F₇, R² is ethyl, R³ is hydrogen, R⁴ is methyl and B for a compound corresponds in each case to one row of Table A

### Table 18

Compounds of the formula I.A in which R¹ is C₃F₇, R² is ethyl, R³ is hydrogen, R⁴ is trifluoromethyl and B for a compound corresponds in each case to one row of Table A

### Table 19

Compounds of the formula I.A in which R¹ is C₄F₉, R² is ethyl, R³ is hydrogen, R⁴ is methyl and B for a compound corresponds in each case to one row of Table A

### Table 20

Compounds of the formula I.A in which R¹ is C₄F₉, R² is ethyl, R³ is hydrogen, R⁴ is trifluoromethyl and B for a compound corresponds in each case to one row of Table A

### Table 21

Compounds of the formula I.A in which R¹ is 4-Cl—C₆H₅, R² is ethyl, R³ is hydrogen, R⁴ is methyl and B for a compound corresponds in each case to one row of Table A

### Table 22

Compounds of the formula I.A in which R¹ is 4-Cl—C₆H₅, R² is ethyl, R³ is hydrogen, R⁴ is trifluoromethyl and B for a compound corresponds in each case to one row of Table A

### Table 23

Compounds of the formula I.A in which R¹ is 4-F—C₆H₅, R² is ethyl, R³ is hydrogen, R⁴ is methyl and B for a compound corresponds in each case to one row of Table A

### Table 24

Compounds of the formula I.A in which R¹ is 4-F—C₆H₅, R² is ethyl, R³ is hydrogen, R⁴ is trifluoromethyl and B for a compound corresponds in each case to one row of Table A

### Table 25

Compounds of the formula I.A in which R¹ is 4-Br—C₆H₅, R² is ethyl, R³ is hydrogen, R⁴ is methyl and B for a compound corresponds in each case to one row of Table A

### Table 26

Compounds of the formula I.A in which R¹ is 4-Br—C₆H₅, R² is ethyl, R³ is hydrogen, R⁴ is trifluoromethyl and B for a compound corresponds in each case to one row of Table A

### Table 27

Compounds of the formula I.A in which R¹ is 4-CH₃—C₆H₅, R² is ethyl, R³ is hydrogen, R⁴ is methyl and B for a compound corresponds in each case to one row of Table A

### Table 28

Compounds of the formula I.A in which R¹ is 4-CH₃—C₆H₅, R² is ethyl, R³ is hydrogen, R⁴ is trifluoromethyl and B for a compound corresponds in each case to one row of Table A

TABLE A

| No. | B |
|---|---|
| A-1 | C₆H₅ |
| A-2 | 2-F—C₆H₄ |
| A-3 | 3-F—C₆H₄ |
| A-4 | 4-F—C₆H₄ |
| A-5 | 2-Cl—C₆H₄ |
| A-6 | 3-Cl—C₆H₄ |
| A-7 | 4-Cl—C₆H₄ |
| A-8 | 2-Br—C₆H₄ |
| A-9 | 3-Br—C₆H₄ |
| A-10 | 4-Br—C₆H₄ |
| A-11 | 2-OH—C₆H₄ |
| A-12 | 3-OH—C₆H₄ |
| A-13 | 4-OH—C₆H₄ |
| A-14 | 2-NO₂—C₆H₄ |
| A-15 | 3-NO₂—C₆H₄ |
| A-16 | 4-NO₂—C₆H₄ |
| A-17 | 2-CN—C₆H₄ |
| A-18 | 3-CN—C₆H₄ |
| A-19 | 4-CN—C₆H₄ |
| A-20 | 2-NH₂—C₆H₄ |
| A-21 | 3-NH₂—C₆H₄ |
| A-22 | 4-NH₂—C₆H₄ |
| A-23 | 2-N(CH₃)₂—C₆H₄ |
| A-24 | 3-N(CH₃)₂—C₆H₄ |
| A-25 | 4-N(CH₃)₂—C₆H₄ |
| A-26 | 2-N(CH₂CH₃)₂—C₆H₄ |
| A-27 | 3-N(CH₂CH₃)₂—C₆H₄ |
| A-28 | 4-N(CH₂CH₃)₂—C₆H₄ |
| A-29 | 2-CH₃—C₆H₄ |
| A-30 | 3-CH₃—C₆H₄ |
| A-31 | 4-CH₃—C₆H₄ |
| A-32 | 2-CH₂CH₃—C₆H₄ |
| A-33 | 3-CH₂CH₃—C₆H₄ |
| A-34 | 4-CH₂CH₃—C₆H₄ |
| A-35 | 2-OCH₃—C₆H₄ |
| A-36 | 3-OCH₃—C₆H₄ |
| A-37 | 4-OCH₃—C₆H₄ |
| A-38 | 2-OCH₂CH₃—C₆H₄ |
| A-39 | 3-OCH₂CH₃—C₆H₄ |
| A-40 | 4-OCH₂CH₃—C₆H₄ |
| A-41 | 2-(O—C₆H₅)—C₆H₄ |
| A-42 | 3-(O—C₆H₅)—C₆H₄ |
| A-43 | 4-(O—C₆H₅)—C₆H₄ |
| A-44 | 2,4-F₂—C₆H₃ |
| A-45 | 3,4-F₂—C₆H₃ |
| A-46 | 3,5-F₂—C₆H₃ |
| A-47 | 2,4-Cl₂—C₆H₃ |
| A-48 | 3,4-Cl₂—C₆H₃ |
| A-49 | 3,5-Cl₂—C₆H₃ |
| A-50 | 2,4-Br₂—C₆H₃ |
| A-51 | 3,4-Br₂—C₆H₃ |
| A-52 | 3,5-Br₂—C₆H₃ |
| A-53 | 2-F-4-Cl—C₆H₃ |
| A-54 | 3-F-4-Cl—C₆H₃ |
| A-55 | 3-F-5-Cl—C₆H₃ |
| A-56 | 2-F-4-Br—C₆H₃ |
| A-57 | 3-F-4-Br—C₆H₃ |
| A-58 | 3-F-5-Br—C₆H₃ |
| A-59 | 2-Cl-4-F—C₆H₃ |
| A-60 | 3-Cl-4-F—C₆H₃ |
| A-61 | 3-Cl-5-F—C₆H₃ |
| A-62 | 2-Cl-4-Br—C₆H₃ |
| A-63 | 2-Cl-4-Br—C₆H₃ |
| A-64 | 3-Cl-5-Br—C₆H₃ |
| A-65 | 2-Br-4-F—C₆H₃ |
| A-66 | 3-Br-4-F—C₆H₃ |
| A-67 | 3-Br-5-F—C₆H₃ |
| A-68 | 2-Br-4-Cl—C₆H₃ |
| A-69 | 3-Br-4-Cl—C₆H₃ |
| A-70 | 3-Br-5-Cl—C₆H₃ |
| A-71 | 2-F-4-CH₃—C₆H₃ |
| A-72 | 3-F-4-CH₃—C₆H₃ |
| A-73 | 3-F-5-CH₃—C₆H₃ |
| A-74 | 2-Cl-4-CH₃—C₆H₃ |
| A-75 | 3-Cl-4-CH₃—C₆H₃ |
| A-76 | 3-Cl-5-CH₃—C₆H₃ |
| A-77 | 2-Br-4-CH₃—C₆H₃ |
| A-78 | 3-Br-4-CH₃—C₆H₃ |
| A-79 | 3-Br-5-CH₃—C₆H₃ |
| A-80 | 3-CH₃-4-F—C₆H₃ |
| A-81 | 3-CH₃-4-Cl—C₆H₃ |
| A-82 | 3-CH₃-4-Br—C₆H₃ |

The compounds I are suitable as fungicides. They are distinguished through an outstanding effectiveness against a broad spectrum of phytopathogenic fungi from the classes of the Ascomycetes, Deuteromycetes, Oomycetes and Basidiomycetes. Some are systemically effective and they can be used in plant protection as foliar fungicides, as fungicides for seed dressing and as soil fungicides.

They are particularly important in the control of a multitude of fungi on various cultivated plants, such as wheat, rye, barley, oats, rice, maize, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on vegetables, oilseed rape, sugar beet and fruit and rice, such as, for example, A. solani or A. alternata on potatoes and tomatoes;
Aphanomyces species on sugar beet and vegetables;
Ascochyta species on cereals and vegetables;
Bipolaris and Drechslera species on corn, cereals, rice and lawns, such as, for example, D. maydis on corn;
Blumeria graminis (powdery mildew) on cereals;
Botrytis cinerea (gray mold) on strawberries, vegetables, flowers and grapevines;
Bremia lactucae on lettuce;
Cercospora species on corn, soybeans, rice and sugar beet;
Cochliobolus species on corn, cereals, rice, such as, for example, Cochliobolus sativus on cereals, Cochliobolus miyabeanus on rice;
Colletotricum species on soybeans and cotton;
Drechslera species, Pyrenophora species on corn, cereals, rice and lawns, such as, for example, D. teres on barley or D. tritici-repentis on wheat;
Esca on grapevines, caused by Phaeoacremonium chlamydosporium, Ph. Aleophilum and Formitipora punctata (syn. Phellinus punctatus);
Exserohilum species on corn;
Erysiphe cichoracearum and Sphaerotheca fuliginea on cucumber plants;
Fusarium and Verticillium species on various plants, such as, for example, F. graminearum or F. culmorum on cereals or F. oxysporum on a multitude of plants, such as, for example, tomatoes;
Gaeumanomyces graminis on cereals;
Gibberella species on cereals and rice (for example Gibberella fujikuroi on rice);
Grainstaining complex on rice;
Helminthosporium species on corn and rice;
Michrodochium nivale on cereals;
Mycosphaerella species on cereals, bananas and groundnuts, such as, for example, M. graminicola on wheat or M. fijiensis on bananas;
Peronospora species on cabbage and bulbous plants, such as, for example, P. brassicae on cabbage or P. destructor on onion;
Phakopsara pachyrhizi and Phakopsara meibomiae on soybeans;
Phomopsis species on soybeans and sunflowers;
Phytophthora infestans on potatoes and tomatoes;
Phytophthora species on various plants, such as, for example, P. capsici on bell pepper;
Plasmopara viticola on grapevines;
Podosphaera leucotricha on apple;
Pseudocercosporella herpotrichoides on cereals;
Pseudoperonospora on various plants, such as, for example, P. cubensis on cucumber or P. humili on hops;
Puccinia species on various plants, such as, for example, P. triticina, P. striformins, P. hordei or P. graminis on cereals or P. asparagi on asparagus;
Pyricularia oryzae, Corticium sasakii, Sarocladium oryzae, S. attenuatum, Entyloma oryzae on rice;
Pyricularia grisea on lawns and cereals;
Pythium spp. on lawns, rice, corn, cotton, oilseed rape, sunflowers, sugar beet, vegetables and other plants, such as, for example, P. ultiumum on various plants, P. aphanidermatum on lawns;
Rhizoctonia species on cotton, rice, potatoes, lawns, corn, oilseed rape, potatoes, sugar beet, vegetables and on various plants, such as, for example, R. solani on beet and various plants;
Rhynchosporium secalis on barley, rye and triticale;
Sclerotinia species on oilseed rape and sunflowers;
Septoria tritici and Stagonospora nodorum on wheat;
Erysiphe (syn. Uncinula) necator on grapevines;
Setospaeria species on corn and lawns;
Sphacelotheca reilinia on corn;
Thievaliopsis species on soybeans and cotton;
Tilletia species on cereals;
Ustilago species on cereals, corn and sugar cane, such as, for example, U. maydis on corn;
Venturia species (scab) on apples and pears, such as, for example, V. inaequalis on apple.

They are particularly suitable for controlling harmful fungi from the class of the Peronosporomycetes (syn. Oomycetes), such as Peronospora species, Phytophthora species, Plasmopara viticola, Pseudoperonospora species and Pythium species.

The compounds I are also suitable for controlling harmful fungi in the protection of materials (for example wood, paper, paint dispersions, fibers or fabrics) and in the protection of stored products. In the protection of wood, particular attention is paid to the following harmful fungi: Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

The compounds I are employed by treating the fungi or the plants, seeds or materials to be protected against fungal attack or the soil with a fungicidally effective amount of the active compounds. Application can be both before and after the infection of the materials, plants or seeds by the fungi.

The fungicidal compositions generally comprise between 0.1 and 95% by weight, preferably between 0.5 and 90% by weight, of active compound.

When employed in crop protection, the application rates are, depending on the kind of effect desired, between 0.01 and 2.0 kg of active compound per ha.

In seed treatment, the amounts of active compound required are generally from 1 to 1000 g/100 kg of seed, preferably from 5 to 100 g/100 kg of seed.

When used in the protection of materials or stored products, the active compound application rates depend on the kind of application area and on the desired effect. Amounts typically applied in the protection of materials are, for example, from 0.001 g to 2 kg, preferably from 0.005 g to 1 kg, of active compound per cubic meter of treated material.

The compounds of the formula I can be present in different crystal modifications which may differ in their biological activity. They are likewise subject matter of the present invention.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries suitable for this purpose are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example finely divided silica, silicates); emulsifiers such as nonionogenic and anionic emulsifiers (for example polyoxyethylene fatty alcohol ethers, alkylsulfonates and arylsulfonates) and dispersants such as lignosulfite waste liquors and methylcellulose.

Suitable for use as surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations: 1. Products for dilution with water

### A Water-Soluble Concentrates (SL, LS)

10 parts by weight of the active compounds are dissolved with 90 parts by weight of water or with a water-soluble solvent. As an alternative, wetters or other auxiliaries are added. The active compound dissolves upon dilution with water. This gives a formulation having an active compound content of 10% by weight.

### B Dispersible Concentrates (DC)

20 parts by weight of the active compounds are dissolved in 70 parts by weight of cyclohexanone with addition of 10 parts by weight of a dispersant, for example polyvinylpyrrolidone. Dilution with water gives a dispersion. The active compound content is 20% by weight

### C Emulsifiable Concentrates (EC)

15 parts by weight of the active compounds are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

### D Emulsions (EW, EO, ES)

25 parts by weight of the active compounds are dissolved in 35 parts by weight of xylene with addition of calcium dodecylbenzenesulfonate and castor oil ethoxylate (in each case 5 parts by weight). This mixture is added to 30 parts by weight of water by means of an emulsifying machine (e.g. Ultraturrax) and made into a homogeneous emulsion. Dilution with water gives an emulsion. The formulation has an active compound content of 25% by weight.

### E Suspensions (SC, OD, FS)

In an agitated ball mill, 20 parts by weight of the active compounds are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

### F Water-Dispersible Granules and Water-Soluble Granules (WG, SG)

50 parts by weight of the active compounds are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active compound. The formulation has an active compound content of 50% by weight.

### G Water-Dispersible Powders and Water-Soluble Powders (WP, SP, SS, WS)

75 parts by weight of the active compounds are ground in a rotor-stator mill with addition of 25 parts by weight of dispersants, wetters and silica gel. Dilution with water gives a stable dispersion or solution of the active compound. The active compound content of the formulation is 75% by weight.

### H Gel Formulations (GF)

20 parts by weight of the active compounds, 10 parts by weight of dispersant, 1 part by weight of gelling agent and 70 parts by weight of water or an organic solvent are ground in a ball mill to give a fine suspension. Dilution with water gives a stable suspension with an active compound content of 20% by weight.

2. Products to be Applied Undiluted

### I Dusts (DP, DS)

5 parts by weight of the active compounds are ground finely and mixed intimately with 95 parts by weight of finely divided kaolin. This gives a dustable product with an active compound content of 5% by weight.

### J Granules (GR, FG, GG, MG)

0.5 part by weight of the active compounds is ground finely and associated with 99.5 parts by weight of carriers. Current methods are extrusion, spray-drying or the fluidized bed. This gives granules with an active compound content of 0.5% by weight to be applied undiluted.

### K ULV Solutions (UL)

10 parts by weight of the active compounds are dissolved in 90 parts by weight of an organic solvent, for example xylene. This gives a product with an active compound content of 10% by weight to be applied undiluted.

Water-soluble concentrates (LS), suspensions (FS), dusts (DS), water-dispersible and water-soluble powders (WS, SS), emulsions (ES), emulsifiable concentrates (EC) and gel formulations (GF) are usually used for the treatment of seed. These formulations can be applied to the seed in undiluted or, preferably, diluted form. The application can be carried out before sowing.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These compositions can be admixed with the compositions according to the invention in a weight ratio of from 1:100 to 100:1, preferably from 1:10 to 10:1.

The following are particularly suitable as adjuvants in this context: organically modified polysiloxanes, for example Break Thru S 240®; alcohol alkoxylates, for example Atplus 245®, Atplus MBA 1303®, Plurafac LF 300® and Lutensol ON 30®; EO-PO block polymers, for example Pluronic RPE 2035® and Genapol B®; alcohol ethoxylates, for example Lutensol XP 80®; and sodium dioctylsulfosuccinate, for example Leophen RA®.

The compositions according to the invention in the application form as fungicides can also be present together with other active compounds, for example with herbicides, insecticides, growth regulators, fungicides or else with fertilizers. When mixing the compounds I or the compositions comprising them with one or more further active compounds, in particular fungicides, it is in many cases possible, for example, to widen the activity spectrum or to prevent the development of resistance. In many cases, synergistic effects are obtained.

The following list of fungicides, with which the compounds according to the invention can be used in conjunction, is intended to illustrate the possible combinations but does not limit them:

### Strobilurins

azoxystrobin, dimoxystrobin, enestroburin, fluoxastrobin, kresoxim-methyl, metominostrobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate, methyl 2-(ortho-(2,5-dimethylphenyloxymethylene)phenyl)-3-methoxyacrylate;

### Carboxamides

carboxanilides: benalaxyl, benodanil, boscalid, carboxin, mepronil, fenfuram, fenhexamid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluorobiphenyl-2-yl)-3-difluoromethyl-1-methylpyrazole-4-carboxamide, N-(2-cyanophenyl)-3,4-dichloroisothiazole-5-carboxamide;
carboxylic acid morpholides: dimethomorph, flumorph;
benzamides: flumetover, fluopicolide (picobenzamid), zoxamide;
other carboxamides: carpropamid, diclocymet, mandipropamid, N-(2-(4-[3-(4-chlorophenyl)prop-2-ynyloxy]-3-methoxyphenyl)ethyl)-2-methanesulfonylamino-3-methylbutyramide, N-(2-(4-[3-(4-chlorophenyl)prop-2-ynyloxy]-3-methoxyphenyl)ethyl)-2-ethanesulfonylamino-3-methylbutyramide;

### Azoles

triazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole;
imidazoles: cyazofamid, imazalil, pefurazoate, prochloraz, triflumizole;
benzimidazoles: benomyl, carbendazim, fuberidazole, thiabendazole;
others: ethaboxam, etridiazole, hymexazole;

### Nitrogenous Heterocyclyl Compounds

pyridines: fluazinam, pyrifenox, 3-[5-(4-chlorophenyl)-2,3-dimethylisoxazolidin-3-yl]-pyridine;
pyrimidines: bupirimate, cyprodinil, ferimzone, fenarimol, mepanipyrim, nuarimol, pyrimethanil;
piperazines: triforine;
pyrroles: fludioxonil, fenpiclonil;
morpholines: aldimorph, dodemorph, fenpropimorph, tridemorph;
dicarboximides: iprodione, procymidone, vinclozolin;
others: acibenzolar-5-methyl, anilazine, captan, captafol, dazomet, diclomezine, fenoxanil, folpet, fenpropidin, famoxadone, fenamidone, octhilinone, probenazole, proquinazid, pyroquilon, quinoxyfen, tricyclazole, 5-chloro-7-(4-methylpiperidin-1-yl)-6-(2,4,6-trifluorophenyl)-[1,2,4]triazolo[1,5-a]pyrimidine, 2-butoxy-6-iodo-3-propylchromen-4-one, N,N-dimethyl-3-(3-bromo-6-fluoro-2-methylindole-1-sulfonyl)-[1,2,4]triazole-1-sulfonamide;

### Carbamates and Dithiocarbamates

dithiocarbamates: ferbam, mancozeb, maneb, metiram, metam, propineb, thiram, zineb, ziram;
carbamates: diethofencarb, flubenthiavalicarb, iprovalicarb, propamocarb, methyl 3-(4-chlorophenyl)-3-(2-isopropoxycarbonylamino-3-methylbutyrylamino)propionate, 4-fluorophenyl N-(1-(1-(4-cyanophenyl)ethanesulfonyl)but-2-yl)carbamate;

### Other Fungicides

guanidines: dodine, iminoctadine, guazatine;
antibiotics: kasugamycin, polyoxins, streptomycin, validamycin A;
organometallic compounds: fentin salts;
sulfur-containing heterocyclyl compounds: isoprothiolane, dithianon;
organophosphorus compounds: edifenphos, fosetyl, fosetyl-aluminum, iprobenfos, pyrazophos, tolclofos-methyl, phosphorous acid and its salts;
organochlorine compounds: thiophanate-methyl, chlorothalonil, dichlofluanid, tolylfluanid, flusulfamide, phthalide, hexachlorobenzene, pencycuron, quintozene;
nitrophenyl derivatives: binapacryl, dinocap, dinobuton;
inorganic active compounds: Bordeaux mixture, copper acetate, copper hydroxide, copper oxychloride, basic copper sulfate, sulfur;
others: spiroxamine, cyflufenamid, cymoxanil, metrafenone.

### SYNTHESIS EXAMPLES

The procedure described in the following synthesis example was used to obtain further compounds I by appropriate modification of the starting materials. The compounds obtained in this manner are listed in the table below, together with physical data.

### Example 1

### Preparation of (4-ethyl-5-hydroxy-3,5-dimethyl-4,5-dihydropyrazol-1-yl)phenylmethanone

A solution of 0.55 g of benzoic acid hydrazide and 0.51 g of ethylpentane-2,4-dione in 40 ml of ethanol was stirred at 20 to 25° C. for about 50 hours. The reaction mixture was freed from the solvent and purified by chromatography on silica gel. This gave 0.4 g of the title compound product as a colorless oil.

TABLE I

| | | | | | | Phys. data (m.p. [° C.]\| |
|---|---|---|---|---|---|---|
| No. | B-A- | R¹ | R² | R³ | R⁴ | ¹H NMR (CDCl₃ δ [pppm] |
| I-1 | C₆H₅—C(═O)-# | CH₃ | CH₂CH₃ | H | CH₃ | 7.85 (d), 7.40 (m), 5.10 (s), 2.95 (m), 2.65 (m), |
| | | | | | | 2.00-1.60 (m), 1.40 (s), 1.10 (m) |
| I-2 | 3-CH₃—C₆H₄—C(═O)-# | CH₃ | CH₂CH₃ | H | CH₃ | 87-90 |
| I-3 | 3-Cl—C₆H₄—C(═O)-# | CH₃ | CH₃ | H | CH₃ | 110-112 |
| I-4 | 3-OH—C₆H₄—C(═O)-# | CH₃ | CH₃ | H | CH₃ | 106-108 |
| I-5 | 4-Cl—C₆H₄—C(═O)-# | CH₃ | CH₂CH₃ | H | CH₃ | 112-113 |
| I-6 | 2-OCH₃, 4-OH—C₆H₃—C(═O)-# | CH₃ | CH₃ | H | CH₃ | 85-94 |
| I-7 | 4-CH₃—C₆H₄—C(═O)-# | CH₃ | CH₃ | H | CH₃ | 7.75 (d), 7.20 (d), 5.10 (s), 5.00 (s), 3.05 (m), |
| | | | | | | 2.80 (m), 2.40 (s), 2.00 (s), 1.85 (s), 1.75 (s), 1.20 (m) |

#denotes the bond to the pyrazoline ring

### Examples of the Action Against Harmful Fungi

The fungicidal action of the compounds of the formula I was demonstrated by the following tests:

The active compounds were prepared as a stock solution comprising 25 mg of active compound which was made up to 10 ml using a mixture of acetone and/or DMSO and the emulsifier Uniperol® EL (wetting agent having emulsifying and dispersing action based on ethoxylated alkylphenols) in a volume ratio of solvent/emulsifier of 99 to 1. The mixture was then made up with water to 100 ml. This stock solution was diluted with the solvent/emulsifier/water mixture described to the concentration of active compounds stated below.

### Use Example 1

### Activity Against Early Blight on Tomatoes Caused by Alternaria solani

Leaves of potted tomato plants were sprayed to runoff point with an aqueous suspension having the concentration of active compounds stated below. The next day, the leaves were infected with an aqueous spore suspension of Alternaria solani in a 2% biomalt solution having a density of 0.17×10⁶ spores/ml. The plants were then placed in a water vapor-saturated chamber at temperatures between 20 and 22° C. After 5 days, the disease on the untreated, but infected control plants had developed to such an extent that the infection could be determined visually in %.

In this test, the plants which had been treated with 250 ppm of the active compound I-2 showed an infection of 20%, whereas the untreated plants were 90% infected.

1-10. (canceled)

11. A method for controlling phytopathogenic harmful fungi comprising:
treating the fungi or materials, plants, soil or seed to be protected against fungal attack with an effective amount of a 5-hydroxypyrazoline of formula I: 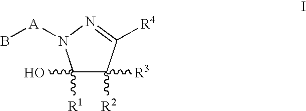
wherein
B is phenyl, naphthyl or 5- or 6-membered hetaryl containing one to four heteroatoms from the group consisting of O, N and S;
A is C═O, C═S or SO₂;
R¹ is C₁-C₁₀-alkyl, C₁-C₁₀-haloalkyl, C₃-C₁₀-alkenyl, C₃-C₁₀-haloalkenyl, C₃-C₁₀-alkynyl, or C₃-C₁₀-haloalkynyl;
C₃-C₁₀-cycloalkyl, C₃-C₁₀-cycloalkenyl, C₈-C₁₀-cycloalkynyl, phenyl, 5- or 6-membered heterocyclyl or hetaryl containing one to four heteroatoms from the group consisting of O, N, and S;
R² is C₁-C₁₀-alkyl, C₁-C₁₀-haloalkyl, C₂-C₁₀-alkenyl, C₂-C₁₀-haloalkyl, C₃-C₁₀-alkynyl, C₃-C₁₀-haloalkynyl, C₃-C₁₀-cycloalkyl, C₃-C₁₀-cycloalkenyl, C₃-C₁₀-cycloalkynyl, phenyl, 5- or 6-membered hetaryl or heterocyclyl containing one to four heteroatoms from the group consisting of O, N, and S;
R³ is hydrogen, nitro, cyano, NR′₂, C₁-C₄-alkyl, C₁-C₄-haloalkyl, C₁-C₄-alkoxy, C₁-C₄-haloalkoxy, C₂-C₄-alkenyl, C₂-C₄-haloalkenyl, C₂-C₄-alkynyl, or C₂-C₄-haloalkynyl, wherein
R′ independently of one another are hydrogen or C₁-C₄-alkyl;
R⁴ is hydrogen, halogen, nitro, cyano, NR′₂, C₁-C₄-alkyl, C₁-C₄-haloalkyl, COOR′, phenyl, 5- or 6-membered hetaryl or heterocyclyl containing one to four heteroatoms from the group consisting of O, N, and S;
wherein the variables mentioned above may be partially or fully halogenated and/or may carry one to four groups R^{a}:
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, formyl, C₁-C₆-alkylthio, C₁-C₆-alkylamino, di-C₁-C₆-alkylamino, C₂-C₈-alkenyl, C₂-C₈-haloalkenyl, C₃-C₈-cycloalkenyl, C₂-C₆-alkenyloxy, C₃-C₆-haloalkenyloxy, C₂-C₆-alkynyl, C₂-C₆-haloalkynyl, C₃-C₆-alkynyloxy, C₃-C₆-haloalkynyloxy, C₃-C₆-cycloalkoxy, C₃-C₆-cycloalkenoxy, C₁-C₃-oxyalkylenoxy, phenyl, naphthyl, a five- to ten-membered saturated, partially unsaturated or aromatic heterocycle containing one to four heteroatoms from the group consisting of O, N, and S;
CRⁱⁱⁱ═NOR^{iv}, wherein
Rⁱⁱⁱ is hydrogen, alkyl, cycloalkyl, or aryl; and
R^{iv} is alkyl, alkenyl, haloalkenyl, alkynyl, or arylalkyl; or
NR^{v}—CO-D-R^{vi}, wherein
R^{v} is hydrogen, hydroxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-alkoxy-C₁-C₆-alkyl, C₁-C₆-alkoxy-C₁-C₆-alkoxy or C₁-C₆-alkoxycarbonyl,
R^{vi} is hydrogen, C₁-C₆-alkyl, C₂-C₆-alkenyl, C₂-C₆-alkynyl, C₃-C₆-cycloalkyl, C₃-C₆-cycloalkenyl, phenyl, phenyl-C₁-C₆-alkyl, hetaryl or hetaryl-C₁-C₆-alkyl, and
D is a direct bond, oxygen or nitrogen, wherein the nitrogen may carry one of the groups R^{vi},
wherein the aliphatic, alicyclic or aromatic groups R^{a} may be partially or fully halogenated or may carry one to three groups R^{b}:
R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, wherein the alkyl groups in these radicals contain 1 to 6 carbon atoms, and the alkenyl or alkynyl groups in these radicals contain 2 to 8 carbon atoms;
and/or one to three of the following radicals:
cycloalkyl, cycloalkoxy, heterocyclyl, heterocyclyloxy, wherein the cyclic systems contain 3 to 10 ring members; phenyl, phenoxy, phenylthio, phenyl-C₁-C₆-alkoxy, phenyl-C₁-C₆-alkyl, hetaryl, hetaryloxy, hetarylthio, wherein the hetaryl groups contain 5 or 6 ring members, and the cyclic systems may be partially or fully halogenated or substituted by alkyl or haloalkyl groups;
wherein phytopathogenic harmful fungi are controlled.

12. A compound of formula I.A: 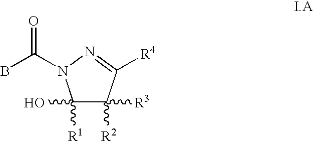
wherein
B is phenyl, naphthyl or 5- or 6-membered hetaryl containing one to four heteroatoms from the group consisting of O, N and S;
R¹ is C₁-C₁₀-alkyl, C₁-C₁₀-haloalkyl, C₃-C₁₀-alkenyl, C₃-C₁₀-haloalkenyl, C₃-C₁₀-alkynyl, or C₃-C₁₀-haloalkynyl;
C₃-C₁₀-cycloalkyl, C₃-C₁₀-cycloalkenyl, C₈-C₁₀-cycloalkynyl, phenyl, 5- or 6-membered heterocyclyl or hetaryl containing one to four heteroatoms from the group consisting of O, N, and S;
R² is C₁-C₁₀-alkyl, C₁-C₁₀-haloalkyl, C₂-C₁₀-alkenyl, C₂-C₁₀-haloalkyl, C₃-C₁₀-alkynyl, C₃-C₁₀-haloalkynyl, C₃-C₁₀-cycloalkyl, C₃-C₁₀-cycloalkenyl, C₃-C₁₀-cycloalkynyl, phenyl, 5- or 6-membered hetaryl or heterocyclyl containing one to four heteroatoms from the group consisting of O, N, and S;
R³ is hydrogen, nitro, cyano, NR′₂, C₁-C₄-alkyl, C₁-C₄-haloalkyl, C₁-C₄-alkoxy, C₁-C₄-haloalkoxy, C₂-C₄-alkenyl, C₂-C₄-haloalkenyl, C₂-C₄-alkynyl, or C₂-C₄-haloalkynyl, wherein
R′ independently of one another are hydrogen or C₁-C₄-alkyl;
R⁴ is hydrogen, halogen, nitro, cyano, NR′₂, C₁-C₄-alkyl, C₁-C₄-haloalkyl, COOR′, phenyl, 5- or 6-membered hetaryl or heterocyclyl containing one to four heteroatoms from the group consisting of O, N, and S;
wherein the variables mentioned above may be partially or fully halogenated and/or may carry one to four groups R^{a}:
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, formyl, C₁-C₆-alkylthio, C₁-C₆-alkylamino, di-C₁-C₆-alkylamino, C₂-C₈-alkenyl, C₂-C₈-haloalkenyl, C₃-C₈-cycloalkenyl, C₂-C₆-alkenyloxy, C₃-C₆-haloalkenyloxy, C₂-C₆-alkynyl, C₂-C₆-haloalkynyl, C₃-C₆-alkynyloxy, C₃-C₆-haloalkynyloxy, C₃-C₆-cycloalkoxy, C₃-C₆-cycloalkenoxy, C₁-C₃-oxyalkylenoxy, phenyl, naphthyl, a five- to ten-membered saturated, partially unsaturated or aromatic heterocycle containing one to four heteroatoms from the group consisting of O, N, and S;
CRⁱⁱⁱ═NOR^{iv}, wherein
Rⁱⁱⁱ is hydrogen, alkyl, cycloalkyl, or aryl; and
R^{iv} is alkyl, alkenyl, haloalkenyl, alkynyl, or arylalkyl; or
NR^{v}—CO-D-R^{vi}, wherein
R^{v} is hydrogen, hydroxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-alkoxy-C₁-C₆-alkyl, C₁-C₆-alkoxy-C₁-C₆-alkoxy or C₁-C₆-alkoxycarbonyl,
R^{vi} is hydrogen, C₁-C₆-alkyl, C₂-C₆-alkenyl, C₂-C₆-alkynyl, C₃-C₆-cycloalkyl, C₃-C₆-cycloalkenyl, phenyl, phenyl-C₁-C₆-alkyl, hetaryl or hetaryl-C₁-C₆-alkyl, and
D is a direct bond, oxygen or nitrogen, wherein the nitrogen may carry one of the groups R^{vi},
wherein the aliphatic, alicyclic or aromatic groups R^{a} may be partially or fully halogenated or may carry one to three groups R^{b}
R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, wherein the alkyl groups in these radicals contain 1 to 6 carbon atoms, and the alkenyl or alkynyl groups in these radicals contain 2 to 8 carbon atoms;
and/or one to three of the following radicals:
cycloalkyl, cycloalkoxy, heterocyclyl, heterocyclyloxy, wherein the cyclic systems contain 3 to 10 ring members; phenyl, phenoxy, phenylthio, phenyl-C₁-C₆-alkoxy, phenyl-C₁-C₆-alkyl, hetaryl, hetaryloxy, hetarylthio, wherein the hetaryl groups contain 5 or 6 ring members, and the cyclic systems may be partially or fully halogenated or substituted by alkyl or haloalkyl groups;
wherein:
R¹ is not methyl;
R¹ is not phenyl or bromomethyl if B is unsubstituted or methyl-substituted phenyl, R², R³, and R⁴ are methyl; and
R¹ is not tert-butyl if B is phenyl, R² and R⁴ are methyl, and R³ is hydrogen.

13. The compound of claim 12, wherein R² is C₁-C₁₀-alkyl or C₁-C₁₀-haloalkyl.

14. A compound of formula I.B1: 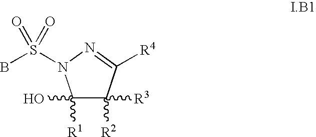
wherein
B is phenyl, naphthyl or 5- or 6-membered hetaryl containing one to four heteroatoms from the group consisting of O, N and S;
R¹ is C₁-C₁₀-alkyl, C₁-C₁₀-haloalkyl, C₃-C₁₀-alkenyl, C₃-C₁₀-haloalkenyl, C₃-C₁₀-alkynyl, or C₃-C₁₀-haloalkynyl;
C₃-C₁₀-cycloalkyl, C₃-C₁₀-cycloalkenyl, C₈-C₁₀-cycloalkynyl, phenyl, 5- or 6-membered heterocyclyl or hetaryl containing one to four heteroatoms from the group consisting of O, N, and S;
R² is C₁-C₁₀-alkyl, C₁-C₁₀-haloalkyl, C₂-C₁₀-alkenyl, C₂-C₁₀-haloalkyl, C₃-C₁₀-alkynyl, C₃-C₁₀-haloalkynyl, C₃-C₁₀-cycloalkyl, C₃-C₁₀-cycloalkenyl, C₃-C₁₀-cycloalkynyl, phenyl, 5- or 6-membered hetaryl or heterocyclyl containing one to four heteroatoms from the group consisting of O, N, and S;
R³ is hydrogen, nitro, cyano, NR′₂, C₁-C₄-alkyl, C₁-C₄-haloalkyl, C₁-C₄-alkoxy, C₁-C₄-haloalkoxy, C₂-C₄-alkenyl, C₂-C₄-haloalkenyl, C₂-C₄-alkynyl, or C₂-C₄-haloalkynyl, wherein
R′ independently of one another are hydrogen or C₁-C₄-alkyl;
R⁴ is hydrogen, halogen, nitro, cyano, NR′₂, C₁-C₄-alkyl, C₁-C₄-haloalkyl, COOR′, phenyl, 5- or 6-membered hetaryl or heterocyclyl containing one to four heteroatoms from the group consisting of O, N, and S;
wherein the variables mentioned above may be partially or fully halogenated and/or may carry one to four groups R^{a}:
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, formyl, C₁-C₆-alkylthio, C₁-C₆-alkylamino, di-C₁-C₆-alkylamino, C₂-C₈-alkenyl, C₂-C₈-haloalkenyl, C₃-C₈-cycloalkenyl, C₂-C₆-alkenyloxy, C₃-C₆-haloalkenyloxy, C₂-C₆-alkynyl, C₂-C₆-haloalkynyl, C₃-C₆-alkynyloxy, C₃-C₆-haloalkynyloxy, C₃-C₆-cycloalkoxy, C₃-C₆-cycloalkenoxy, C₁-C₃-oxyalkylenoxy, phenyl, naphthyl, a five- to ten-membered saturated, partially unsaturated or aromatic heterocycle containing one to four heteroatoms from the group consisting of O, N, and S;
CRⁱⁱⁱ═NOR^{iv}, wherein
Rⁱⁱⁱ is hydrogen, alkyl, cycloalkyl, or aryl; and
R^{iv} is alkyl, alkenyl, haloalkenyl, alkynyl, or arylalkyl; or
NR^{v}—CO-D-R^{vi}, wherein
R^{v} is hydrogen, hydroxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-alkoxy-C₁-C₆-alkyl, C₁-C₆-alkoxy-C₁-C₆-alkoxy or C₁-C₆-alkoxycarbonyl,
R^{vi} is hydrogen, C₁-C₆-alkyl, C₂-C₆-alkenyl, C₂-C₆-alkynyl, C₃-C₆-cycloalkyl, C₃-C₆-cycloalkenyl, phenyl, phenyl-C₁-C₆-alkyl, hetaryl or hetaryl-C₁-C₆-alkyl, and
D is a direct bond, oxygen or nitrogen, wherein the nitrogen may carry one of the groups R^{vi},
wherein the aliphatic, alicyclic or aromatic groups R^{a} may be partially or fully halogenated or may carry one to three groups R^{b}:
R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, wherein the alkyl groups in these radicals contain 1 to 6 carbon atoms, and the alkenyl or alkynyl groups in these radicals contain 2 to 8 carbon atoms;
and/or one to three of the following radicals:
cycloalkyl, cycloalkoxy, heterocyclyl, heterocyclyloxy, wherein the cyclic systems contain 3 to 10 ring members; phenyl, phenoxy, phenylthio, phenyl-C₁-C₆-alkoxy, phenyl-C₁-C₆-alkyl, hetaryl, hetaryloxy, hetarylthio, wherein the hetaryl groups contain 5 or 6 ring members, and the cyclic systems may be partially or fully halogenated or substituted by alkyl or haloalkyl groups.

15. The compound of claim 14, wherein R² is C₁-C₁₀-alkyl or C₁-C₁₀-haloalkyl.

16. A compound of formula I.B2: 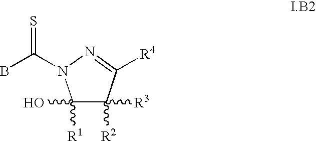
wherein
B is phenyl, naphthyl or 5- or 6-membered hetaryl containing one to four heteroatoms from the group consisting of O, N and S;
R¹ is C₁-C₁₀-alkyl, C₁-C₁₀-haloalkyl, C₃-C₁₀-alkenyl, C₃-C₁₀-haloalkenyl, C₃-C₁₀-alkynyl, or C₃-C₁₀-haloalkynyl;
C₃-C₁₀-cycloalkyl, C₃-C₁₀-cycloalkenyl, C₈-C₁₀-cycloalkynyl, phenyl, 5- or 6-membered heterocyclyl or hetaryl containing one to four heteroatoms from the group consisting of O, N, and S;
R² is C₁-C₁₀-alkyl, C₁-C₁₀-haloalkyl, C₂-C₁₀-alkenyl, C₂-C₁₀-haloalkyl, C₃-C₁₀-alkynyl, C₃-C₁₀-haloalkynyl, C₃-C₁₀-cycloalkyl, C₃-C₁₀-cycloalkenyl, C₃-C₁₀-cycloalkynyl, phenyl, 5- or 6-membered hetaryl or heterocyclyl containing one to four heteroatoms from the group consisting of O, N, and S;
R³ is hydrogen, nitro, cyano, NR′₂, C₁-C₄-alkyl, C₁-C₄-haloalkyl, C₁-C₄-alkoxy, C₁-C₄-haloalkoxy, C₂-C₄-alkenyl, C₂-C₄-haloalkenyl, C₂-C₄-alkynyl, or C₂-C₄-haloalkynyl, wherein
R′ independently of one another are hydrogen or C₁-C₄-alkyl;
R⁴ is hydrogen, halogen, nitro, cyano, NR′₂, C₁-C₄-alkyl, C₁-C₄-haloalkyl, COOR′, phenyl, 5- or 6-membered hetaryl or heterocyclyl containing one to four heteroatoms from the group consisting of O, N, and S;
wherein the variables mentioned above may be partially or fully halogenated and/or may carry one to four groups R^{a}:
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, formyl, C₁-C₆-alkylthio, C₁-C₆-alkylamino, di-C₁-C₆-alkylamino, C₂-C₈-alkenyl, C₂-C₈-haloalkenyl, C₃-C₈-cycloalkenyl, C₂-C₆-alkenyloxy, C₃-C₆-haloalkenyloxy, C₂-C₆-alkynyl, C₂-C₆-haloalkynyl, C₃-C₆-alkynyloxy, C₃-C₆-haloalkynyloxy, C₃-C₆-cycloalkoxy, C₃-C₆-cycloalkenoxy, C₁-C₃-oxyalkylenoxy, phenyl, naphthyl, a five- to ten-membered saturated, partially unsaturated or aromatic heterocycle containing one to four heteroatoms from the group consisting of O, N, and S;
CRⁱⁱⁱ═NOR^{iv}, wherein
Rⁱⁱⁱ is hydrogen, alkyl, cycloalkyl, or aryl; and
R^{iv} is alkyl, alkenyl, haloalkenyl, alkynyl, or arylalkyl; or
NR^{v}—CO-D-R^{vi}, wherein
R^{v} is hydrogen, hydroxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-alkoxy-C₁-C₆-alkyl, C₁-C₆-alkoxy-C₁-C₆-alkoxy or C₁-C₆-alkoxycarbonyl,
R^{vi} is hydrogen, C₁-C₆-alkyl, C₂-C₆-alkenyl, C₂-C₆-alkynyl, C₃-C₆-cycloalkyl, C₃-C₆-cycloalkenyl, phenyl, phenyl-C₁-C₆-alkyl, hetaryl or hetaryl-C₁-C₆-alkyl, and
D is a direct bond, oxygen or nitrogen, wherein the nitrogen may carry one of the groups R^{vi},
wherein the aliphatic, alicyclic or aromatic groups R^{a} may be partially or fully halogenated or may carry one to three groups R^{b}:
R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, wherein the alkyl groups in these radicals contain 1 to 6 carbon atoms, and the alkenyl or alkynyl groups in these radicals contain 2 to 8 carbon atoms;
and/or one to three of the following radicals:
cycloalkyl, cycloalkoxy, heterocyclyl, heterocyclyloxy, wherein the cyclic systems contain 3 to 10 ring members; phenyl, phenoxy, phenylthio, phenyl-C₁-C₆-alkoxy, phenyl-C₁-C₆-alkyl, hetaryl, hetaryloxy, hetarylthio, wherein the hetaryl groups contain 5 or 6 ring members, and the cyclic systems may be partially or fully halogenated or substituted by alkyl or haloalkyl groups;
wherein R¹ is not methyl.

17. The compound claim 16, wherein R² is C₁-C₁₀-alkyl or C₁-C₁₀-haloalkyl.

18. A process for preparing a compound of formula I.A: 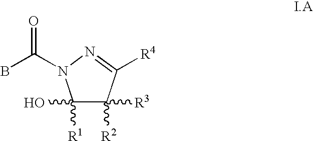
comprising:
contacting a hydrazine of the formula II: 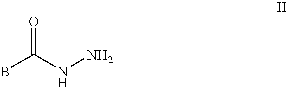
wherein B is phenyl, naphthyl or 5- or 6-membered hetaryl containing one to four heteroatoms from the group consisting of O, N, and S; and
a diketone of the formula III: 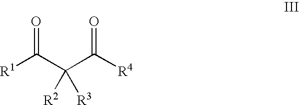
wherein:
R¹ is C₁-C₁₀-alkyl, C₁-C₁₀-haloalkyl, C₃-C₁₀-alkenyl, C₃-C₁₀-haloalkenyl, C₃-C₁₀-alkynyl, or C₃-C₁₀-haloalkynyl;
C₃-C₁₀-cycloalkyl, C₃-C₁₀-cycloalkenyl, C₈-C₁₀-cycloalkynyl, phenyl, 5- or 6-membered heterocyclyl or hetaryl containing one to four heteroatoms from the group consisting of O, N, and S;
R² is C₁-C₁₀-alkyl, C₁-C₁₀-haloalkyl, C₂-C₁₀-alkenyl, C₂-C₁₀-haloalkyl, C₃-C₁₀-alkynyl, C₃-C₁₀-haloalkynyl, C₃-C₁₀-cycloalkyl, C₃-C₁₀-cycloalkenyl, C₃-C₁₀-cycloalkynyl, phenyl, 5- or 6-membered hetaryl or heterocyclyl containing one to four heteroatoms from the group consisting of O, N, and S;
R³ is hydrogen, nitro, cyano, NR′₂, C₁-C₄-alkyl, C₁-C₄-haloalkyl, C₁-C₄-alkoxy, C₁-C₄-haloalkoxy, C₂-C₄-alkenyl, C₂-C₄-haloalkenyl, C₂-C₄-alkynyl, or C₂-C₄-haloalkynyl, wherein
R′ independently of one another are hydrogen or C₁-C₄-alkyl;
R⁴ is hydrogen, halogen, nitro, cyano, NR′₂, C₁-C₄-alkyl, C₁-C₄-haloalkyl, COOR′, phenyl, 5- or 6-membered hetaryl or heterocyclyl containing one to four heteroatoms from the group consisting of O, N, and S;
wherein the variables mentioned above may be partially or fully halogenated and/or may carry one to four groups R^{a}:
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, formyl, C₁-C₆-alkylthio, C₁-C₆-alkylamino, di-C₁-C₆-alkylamino, C₂-C₈-alkenyl, C₂-C₈-haloalkenyl, C₃-C₈-cycloalkenyl, C₂-C₆-alkenyloxy, C₃-C₆-haloalkenyloxy, C₂-C₆-alkynyl, C₂-C₆-haloalkynyl, C₃-C₆-alkynyloxy, C₃-C₆-haloalkynyloxy, C₃-C₆-cycloalkoxy, C₃-C₆-cycloalkenoxy, C₁-C₃-oxyalkylenoxy, phenyl, naphthyl, a five- to ten-membered saturated, partially unsaturated or aromatic heterocycle containing one to four heteroatoms from the group consisting of O, N, and S;
CRⁱⁱⁱ═NOR^{iv}, wherein
Rⁱⁱⁱ is hydrogen, alkyl, cycloalkyl, or aryl; and
R^{iv} is alkyl, alkenyl, haloalkenyl, alkynyl, or arylalkyl; or
NR^{vi}—CO-D-R^{vi}, wherein
R^{v} is hydrogen, hydroxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-alkoxy-C₁-C₆-alkyl, C₁-C₆-alkoxy-C₁-C₆-alkoxy or C₁-C₆-alkoxycarbonyl,
R^{vi} is hydrogen, C₁-C₆-alkyl, C₂-C₆-alkenyl, C₂-C₆-alkynyl, C₃-C₆-cycloalkyl, C₃-C₆-cycloalkenyl, phenyl, phenyl-C₁-C₆-alkyl, hetaryl or hetaryl-C₁-C₆-alkyl, and
D is a direct bond, oxygen or nitrogen, wherein the nitrogen may carry one of the groups R^{vi},
wherein the aliphatic, alicyclic or aromatic groups R^{a} may be partially or fully halogenated or may carry one to three groups R^{b}:
R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, wherein the alkyl groups in these radicals contain 1 to 6 carbon atoms, and the alkenyl or alkynyl groups in these radicals contain 2 to 8 carbon atoms;
and/or one to three of the following radicals:
cycloalkyl, cycloalkoxy, heterocyclyl, heterocyclyloxy, wherein the cyclic systems contain 3 to 10 ring members; phenyl, phenoxy, phenylthio, phenyl-C₁-C₆-alkoxy, phenyl-C₁-C₆-alkyl, hetaryl, hetaryloxy, hetarylthio, wherein the hetaryl groups contain 5 or 6 ring members, and the cyclic systems may be partially or fully halogenated or substituted by alkyl or haloalkyl groups;
wherein:
R¹ is not methyl;
R¹ is not phenyl or bromomethyl if B is unsubstituted or methyl-substituted phenyl,
R², R³, and R⁴ are methyl; and
R¹ is not tert-butyl if B is phenyl, R² and R⁴ are methyl, and R³ is hydrogen;
wherein aa compound of formula I.A is prepared.

19. A fungicidal composition comprising:
a solid or liquid carrier; and
a compound of formula I: 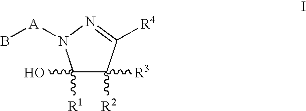
wherein
B is phenyl, naphthyl or 5- or 6-membered hetaryl containing one to four heteroatoms from the group consisting of O, N, and S;
A is C═O, C═S or SO₂;
R¹ is C₁-C₁₀-alkyl, C₁-C₁₀-haloalkyl, C₃-C₁₀-alkenyl, C₃-C₁₀-haloalkenyl, C₃-C₁₀-alkynyl, or C₃-C₁₀-haloalkynyl;
C₃-C₁₀-cycloalkyl, C₃-C₁₀-cycloalkenyl, C₈-C₁₀-cycloalkynyl, phenyl, 5- or 6-membered heterocyclyl or hetaryl containing one to four heteroatoms from the group consisting of O, N, and S;
R² is C₁-C₁₀-alkyl, C₁-C₁₀-haloalkyl, C₂-C₁₀-alkenyl, C₂-C₁₀-haloalkyl, C₃-C₁₀-alkynyl, C₃-C₁₀-haloalkynyl, C₃-C₁₀-cycloalkyl, C₃-C₁₀-cycloalkenyl, C₃-C₁₀-cycloalkynyl, phenyl, 5- or 6-membered hetaryl or heterocyclyl containing one to four heteroatoms from the group consisting of O, N, and S;
R³ is hydrogen, nitro, cyano, NR′₂, C₁-C₄-alkyl, C₁-C₄-haloalkyl, C₁-C₄-alkoxy, C₁-C₄-haloalkoxy, C₂-C₄-alkenyl, C₂-C₄-haloalkenyl, C₂-C₄-alkynyl, or C₂-C₄-haloalkynyl, wherein
R′ independently of one another are hydrogen or C₁-C₄-alkyl;
R⁴ is hydrogen, halogen, nitro, cyano, NR′₂, C₁-C₄-alkyl, C₁-C₄-haloalkyl, COOR′, phenyl, 5- or 6-membered hetaryl or heterocyclyl containing one to four heteroatoms from the group consisting of O, N, and S;
wherein the variables mentioned above may be partially or fully halogenated and/or may carry one to four groups R^{a},
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, formyl, C₁-C₆-alkylthio, C₁-C₆-alkylamino, di-C₁-C₆-alkylamino, C₂-C₈-alkenyl, C₂-C₈-haloalkenyl, C₃-C₈-cycloalkenyl, C₂-C₆-alkenyloxy, C₃-C₆-haloalkenyloxy, C₂-C₆-alkynyl, C₂-C₆-haloalkynyl, C₃-C₆-alkynyloxy, C₃-C₆-haloalkynyloxy, C₃-C₆-cycloalkoxy, C₃-C₆-cycloalkenoxy, C₁-C₃-oxyalkylenoxy, phenyl, naphthyl, a five- to ten-membered saturated, partially unsaturated or aromatic heterocycle containing one to four heteroatoms from the group consisting of O, N, and S,
CRⁱⁱⁱ═NOR^{iv}, wherein
Rⁱⁱⁱ is hydrogen, alkyl, cycloalkyl, or aryl; and
R^{iv} is alkyl, alkenyl, haloalkenyl, alkynyl, or arylalkyl; or
NR^{v}—CO-D-R^{vi}, wherein
R^{v} is hydrogen, hydroxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-alkoxy-C₁-C₆-alkyl, C₁-C₆-alkoxy-C₁-C₆-alkoxy or C₁-C₆-alkoxycarbonyl,
R^{vi} is hydrogen, C₁-C₆-alkyl, C₂-C₆-alkenyl, C₂-C₆-alkynyl, C₃-C₆-cycloalkyl, C₃-C₆-cycloalkenyl, phenyl, phenyl-C₁-C₆-alkyl, hetaryl or hetaryl-C₁-C₆-alkyl, and
D is a direct bond, oxygen or nitrogen, wherein the nitrogen may carry one of the groups R^{vi},
wherein the aliphatic, alicyclic or aromatic groups R^{a} may be partially or fully halogenated or may carry one to three groups R^{b}:
R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, wherein the alkyl groups in these radicals contain 1 to 6 carbon atoms, and the alkenyl or alkynyl groups in these radicals contain 2 to 8 carbon atoms;
and/or one to three of the following radicals:
cycloalkyl, cycloalkoxy, heterocyclyl, heterocyclyloxy, wherein the cyclic systems contain 3 to 10 ring members; phenyl, phenoxy, phenylthio, phenyl-C₁-C₆-alkoxy, phenyl-C₁-C₆-alkyl, hetaryl, hetaryloxy, hetarylthio, wherein the hetaryl groups contain 5 or 6 ring members, and the cyclic systems may be partially or fully halogenated or substituted by alkyl or haloalkyl groups.

20. The fungicidal composition of claim 19, wherein the compound of formula I is selected from the group consisting of: 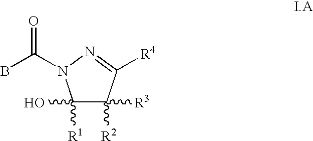
wherein
R¹ of formula I.A is not methyl;
R¹ of formula I.A is not phenyl or bromomethyl if B is unsubstituted or methyl-substituted phenyl, R², R³, and R⁴ are methyl;
R¹ of formula I.A is not tert-butyl if B is phenyl, R² and R⁴ are methyl, and R³ is hydrogen; 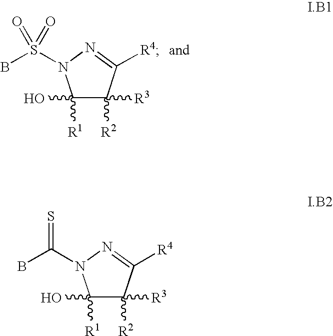
wherein R¹ of formula I.B2 is not methyl.

21. Seed comprising a compound of formula I: 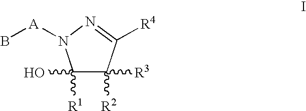
wherein
B is phenyl, naphthyl or 5- or 6-membered hetaryl containing one to four heteroatoms from the group consisting of O, N, and S;
A is C═O, C═S or SO₂;
R¹ is C₁-C₁₀-alkyl, C₁-C₁₀-haloalkyl, C₃-C₁₀-alkenyl, C₃-C₁₀-haloalkenyl, C₃-C₁₀-alkynyl, or C₃-C₁₀-haloalkynyl;
C₃-C₁₀-cycloalkyl, C₃-C₁₀-cycloalkenyl, C₈-C₁₀-cycloalkynyl, phenyl, 5- or 6-membered heterocyclyl or hetaryl containing one to four heteroatoms from the group consisting of O, N, and S;
R² is C₁-C₁₀-alkyl, C₁-C₁₀-haloalkyl, C₂-C₁₀alkenyl, C₂-C₁₀-haloalkyl, C₃-C₁₀-alkynyl, C₃-C₁₀-haloalkynyl, C₃-C₁₀-cycloalkyl, C₃-C₁₀-cycloalkenyl, C₃-C₁₀-cycloalkynyl, phenyl, 5- or 6-membered hetaryl or heterocyclyl containing one to four heteroatoms from the group consisting of O, N, and S;
R³ is hydrogen, nitro, cyano, NR′₂, C₁-C₄-alkyl, C₁-C₄-haloalkyl, C₁-C₄-alkoxy, C₁-C₄-haloalkoxy, C₂-C₄-alkenyl, C₂-C₄-haloalkenyl, C₂-C₄-alkynyl, or C₂-C₄-haloalkynyl, wherein
R¹ independently of one another are hydrogen or C₁-C₄-alkyl;
R⁴ is hydrogen, halogen, nitro, cyano, NR′₂, C₁-C₄-alkyl, C₁-C₄-haloalkyl, COOR′, phenyl, 5- or 6-membered hetaryl or heterocyclyl containing one to four heteroatoms from the group consisting of O, N, and S;
wherein the variables mentioned above may be partially or fully halogenated and/or may carry one to four groups R^{a},
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, formyl, C₁-C₆-alkylthio, C₁-C₆-alkylamino, di-C₁-C₆-alkylamino, C₂-C₈-alkenyl, C₂-C₈-haloalkenyl, C₃-C₈-cycloalkenyl, C₂-C₆-alkenyloxy, C₃-C₆-haloalkenyloxy, C₂-C₆-alkynyl, C₂-C₆-haloalkynyl, C₃-C₆-alkynyloxy, C₃-C₆-haloalkynyloxy, C₃-C₆-cycloalkoxy, C₃-C₆-cycloalkenoxy, C₁-C₃-oxyalkylenoxy, phenyl, naphthyl, a five- to ten-membered saturated, partially unsaturated or aromatic heterocycle containing one to four heteroatoms from the group consisting of O, N, and S,
CRⁱⁱⁱ═NOR^{iv}, wherein
Rⁱⁱⁱ is hydrogen, alkyl, cycloalkyl, or aryl; and
R^{iv} is alkyl, alkenyl, haloalkenyl, alkynyl, or arylalkyl; or
NR^{v}—CO-D-R^{vi}, wherein
R^{v} is hydrogen, hydroxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-alkoxy-C₁-C₆-alkyl, C₁-C₆-alkoxy-C₁-C₆-alkoxy or C₁-C₆-alkoxycarbonyl,
R^{vi} is hydrogen, C₁-C₆-alkyl, C₂-C₆-alkenyl, C₂-C₆-alkynyl, C₃-C₆-cycloalkyl, C₃-C₆-cycloalkenyl, phenyl, phenyl-C₁-C₆-alkyl, hetaryl or hetaryl-C₁-C₆-alkyl, and
D is a direct bond, oxygen or nitrogen, wherein the nitrogen may carry one of the groups R^{vi},
wherein the aliphatic, alicyclic or aromatic groups R^{a} may be partially or fully halogenated or may carry one to three groups R^{b}:
R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, wherein the alkyl groups in these radicals contain 1 to 6 carbon atoms and the alkenyl or alkynyl groups in these radicals contain 2 to 8 carbon atoms;
and/or one to three of the following radicals:
cycloalkyl, cycloalkoxy, heterocyclyl, heterocyclyloxy, wherein the cyclic systems contain 3 to 10 ring members; phenyl, phenoxy, phenylthio, phenyl-C₁-C₆-alkoxy, phenyl-C₁-C₆-alkyl, hetaryl, hetaryloxy, hetarylthio, wherein the hetaryl groups contain 5 or 6 ring members, and the cyclic systems may be partially or fully halogenated or substituted by alkyl or haloalkyl groups;
in an amount of from 1 to 1000 g per 100 kg of seed.

22. The seed composition of claim 21, wherein the compound of formula I is selected from the group consisting of: 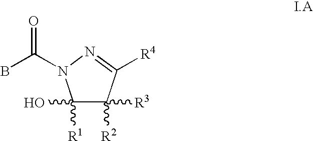
wherein
R¹ of formula I.A is not methyl;
R¹ of formula I.A is not phenyl or bromomethyl if B is unsubstituted or methyl-substituted phenyl, R², R³, and R⁴ are methyl;
R¹ of formula I.A is not tert-butyl if B is phenyl, R² and R⁴ are methyl, and R³ is hydrogen; 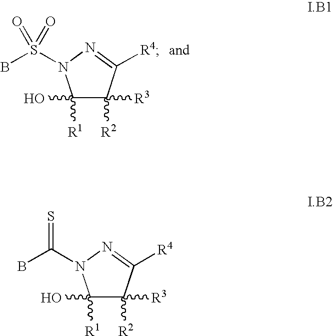
wherein R¹ of formula I.B2 is not methyl.

23. A method of preparing a fungicidal composition comprising:
contacting a solid or liquid carrier with a compound of formula I: 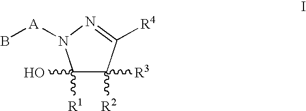
wherein
B is phenyl, naphthyl or 5- or 6-membered hetaryl containing one to four heteroatoms from the group consisting of O, N, and S;
A is C═O, C═S or SO₂;
R¹ is C₁-C₁₀-alkyl, C₁-C₁₀-haloalkyl, C₃-C₁₀-alkenyl, C₃-C₁₀-haloalkenyl, C₃-C₁₀-alkynyl, or C₃-C₁₀-haloalkynyl;
C₃-C₁₀-cycloalkyl, C₃-C₁₀-cycloalkenyl, C₈-C₁₀-cycloalkynyl, phenyl, 5- or 6-membered heterocyclyl or hetaryl containing one to four heteroatoms from the group consisting of O, N, and S;
R² is C₁-C₁₀-alkyl, C₁-C₁₀-haloalkyl, C₂-C₁₀-alkenyl, C₂-C₁₀-haloalkyl, C₃-C₁₀-alkynyl, C₃-C₁₀-haloalkynyl, C₃-C₁₀-cycloalkyl, C₃-C₁₀-cycloalkenyl, C₃-C₁₀-cycloalkynyl, phenyl, 5- or 6-membered hetaryl or heterocyclyl containing one to four heteroatoms from the group consisting of O, N, and S;
R³ is hydrogen, nitro, cyano, NR′₂, C₁-C₄-alkyl, C₁-C₄-haloalkyl, C₁-C₄-alkoxy, C₁-C₄-haloalkoxy, C₂-C₄-alkenyl, C₂-C₄-haloalkenyl, C₂-C₄-alkynyl, or C₂-C₄-haloalkynyl, wherein
R¹ independently of one another are hydrogen or C₁-C₄-alkyl;
R⁴ is hydrogen, halogen, nitro, cyano, NR′₂, C₁-C₄-alkyl, C₁-C₄-haloalkyl, COOR′, phenyl, 5- or 6-membered hetaryl or heterocyclyl containing one to four heteroatoms from the group consisting of O, N, and S;
wherein the variables mentioned above may be partially or fully halogenated and/or may carry one to four groups R^{a},
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, formyl, C₁-C₆-alkylthio, C₁-C₆-alkylamino, di-C₁-C₆-alkylamino, C₂-C₈-alkenyl, C₂-C₈-haloalkenyl, C₃-C₈-cycloalkenyl, C₂-C₆-alkenyloxy, C₃-C₆-haloalkenyloxy, C₂-C₆-alkynyl, C₂-C₆-haloalkynyl, C₃-C₆-alkynyloxy, C₃-C₆-haloalkynyloxy, C₃-C₆-cycloalkoxy, C₃-C₆-cycloalkenoxy, C₁-C₃-oxyalkylenoxy, phenyl, naphthyl, a five- to ten-membered saturated, partially unsaturated or aromatic heterocycle containing one to four heteroatoms from the group consisting of O, N, and S,
CRⁱⁱⁱ═NOR^{iv}, wherein
Rⁱⁱⁱ is hydrogen, alkyl, cycloalkyl, or aryl; and
R^{iv} is alkyl, alkenyl, haloalkenyl, alkynyl, or arylalkyl; or
NR^{v}—CO-D-R^{vi}, wherein
R^{v} is hydrogen, hydroxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-alkoxy-C₁-C₆-alkyl, C₁-C₆-alkoxy-C₁-C₆-alkoxy or C₁-C₆-alkoxycarbonyl,
R^{vi} is hydrogen, C₁-C₆-alkyl, C₂-C₆-alkenyl, C₂-C₆-alkynyl, C₃-C₆-cycloalkyl, C₃-C₆-cycloalkenyl, phenyl, phenyl-C₁-C₆-alkyl, hetaryl or hetaryl-C₁-C₆-alkyl, and
D is a direct bond, oxygen or nitrogen, wherein the nitrogen may carry one of the groups R^{vi},
wherein the aliphatic, alicyclic or aromatic groups R^{a} may be partially or fully halogenated or may carry one to three groups R^{b}:
R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, wherein the alkyl groups in these radicals contain 1 to 6 carbon atoms, and the alkenyl or alkynyl groups in these radicals contain 2 to 8 carbon atoms;
and/or one to three of the following radicals:
cycloalkyl, cycloalkoxy, heterocyclyl, heterocyclyloxy, wherein the cyclic systems contain 3 to 10 ring members; phenyl, phenoxy, phenylthio, phenyl-C₁-C₆-alkoxy, phenyl-C₁-C₆-alkyl, hetaryl, hetaryloxy, hetarylthio, wherein the hetaryl groups contain 5 or 6 ring members, and the cyclic systems may be partially or fully halogenated or substituted by alkyl or haloalkyl groups;
wherein a fungicidal composition is prepared.

24. The method of claim 11, wherein the compound of formula I is selected from the group consisting of: 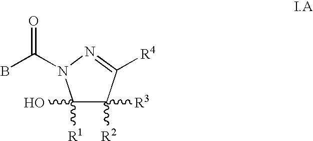
wherein
R¹ of formula I.A is not methyl;
R¹ of formula I.A is not phenyl or bromomethyl if B is unsubstituted or methyl-substituted phenyl, R², R³, and R⁴ are methyl;
R¹ of formula I.A is not tert-butyl if B is phenyl, R² and R⁴ are methyl, and R³ is hydrogen; 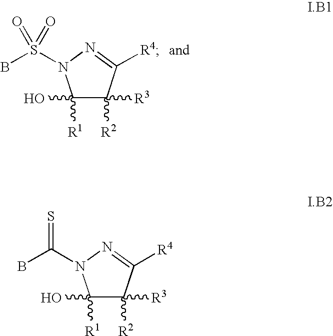
wherein R¹ of formula I.B2 is not methyl.
